(19) United States
(12) Patent Application Publication
(10) Pub. No.: US 2024/0024167 A1
(43) Pub. Date: Jan. 25, 2024

(54) ADJUSTABLE ABSORBENT ARTICLE

(71) Applicant: PRODUCTOS FAMILIA S.A., Medellin (CO)

(72) Inventors: Luisa Fernanda ARBELÁEZ CORREA, Medellín (CO); Fabio Alberto LÓPEZ PIMIENTA, Medellin (CO); Mateo MORALES GOMEZ, Medellín (CO)

(21) Appl. No.: 18/446,455

(22) Filed: Aug. 8, 2023

Related U.S. Application Data
(62) Division of application No. 16/843,196, filed on Apr. 8, 2020.

(30) Foreign Application Priority Data
Apr. 9, 2019 (CO) NC2019/0003528

(51) Int. Cl. A61F 13/474; A61F 13/472; A61F 13/15; A61F 13/531

(52) U.S. Cl. CPC A61F 13/474; A61F 13/47236; A61F 13/15203; A61F 13/531; A61F 2013/15243; A61F 2013/4706

(57) ABSTRACT

The present invention relates to an adjustable absorbent article comprising a top sheet, a bottom sheet, an absorbent core located between the top sheet and the bottom sheet, the absorbent core having two longitudinal ends. Said absorbent core has an oblong shape, wherein one of the longitudinal ends has a triangular shape and an absorbent panel overlapped on the periphery of the triangular-shaped longitudinal end of the absorbent core. Said absorbent panel folds to fit different types of garments. Said adjustable absorbent article also comprises an absorbent core with a section corresponding to an absorbent section, which extends from the periphery of the triangular-shaped longitudinal end towards the exterior of the absorbent article, wherein said absorbent section folds to fit different types of garments.

### CROSS-REFERENCE TO RELATED APPLICATION

This application is a divisional from co-pending application Ser. No. 16/843,196 filed on Apr. 8, 2020, which is hereby incorporated by reference in its entirety.

### FIELD OF THE INVENTION

The present invention is related to absorbent articles. Specifically, the present invention is related to absorbent articles for personal care, which can be adjusted to different types of garments.

### BACKGROUND OF THE INVENTION

Among the different types of personal care products on the market, are those commonly used for the retention or capture of bodily fluids. Said products called absorbent articles, such as sanitary napkins, daily liners and incontinence absorbents differ in shape, size, manufacturing process and absorption capacity, and each of these fulfills a specific function depending on the type of garment in which it is used.

Absorbent articles have specific shapes for each type of garment, so the user should have different absorbent articles that fit each of them. According to the above, sometimes this is not practical for the user, given the variety of different absorbent articles that must be purchased.

Therefore, some ways of solving this problem have been disclosed. U.S. Pat. No. 4,687,478A discloses an absorbent article which comprises an absorbent core, two wings with a wing absorbent core, a topsheet and a bottom sheet which contain the absorbent core and the absorbent wing core, and wherein the wings are flexible, allowing them to fold in such a way that they can be adjusted to different types of garments.

Said wings have a first and a second axis of flexibility, where said axes of flexibility allow the wings to fold to form a barrier against the flow of menstrual fluid. Additionally, the wings have adhesive fastening means located at the distal ends of the wings and in the central part of the absorbent core, the above in order to hold the absorbent article in an undergarment. However, U.S. Pat. No. 4,687,478A does not teach how the absorbent article fits at its ends to different types of garments.

On one hand, US 20030208177A1 discloses an absorbent article with a trapezoidal or triangular shape comprised of an absorbent core, a top sheet and a bottom sheet that contain the absorbent core and a pair of absorbent longitudinal ends on the periphery of the absorbent article. However, US 20030208177A1 does not disclose how to adapt the shape of a single absorbent article in order to fit in different types of underwear.

Finally, U.S. Pat. No. 8,105,302 B2 discloses an adjustable absorbent article comprised of different absorbent panels joined together which have the same thickness. Said panels are joined by seams, allowing the different panels that make up the absorbent article of U.S. Pat. No. 8,105,302 B2, to be folded in order to adjust to different types of underwear. However, the absorbent article disclosed in U.S. Pat. No. 8,105,302 B2 is set up by means of specific geometric shapes, which does not allow it to adequately adapt to different types of garments.

Therefore, while the aforementioned documents disclose adjustable absorbent articles, the prior art cited does not teach how to adapt a single absorbent article to different types of underwear shapes without affecting the absorbent capacity of absorbent articles and without spillage.

### BRIEF DESCRIPTION OF THE INVENTION

The present invention corresponds to an adjustable absorbent article comprising a topsheet, a bottom sheet, an absorbent core located between the top sheet and the bottom sheet, the absorbent core having two longitudinal ends. Said absorbent core has an oblong shape, where one of the longitudinal ends has a triangular shape and an absorbent panel overlapped on the periphery of the triangular shaped longitudinal end of the absorbent core. This absorbent panel folds to fit different types of garments.

Said adjustable absorbent article also comprises an absorbent core with a section corresponding to an absorbent section. Said absorbent section is an extension of the absorbent core, which extends from the periphery of the triangular shaped longitudinal end towards the exterior of the absorbent article, wherein said absorbent section folds to fit different types of garments.

### BRIEF DESCRIPTION OF THE FIGURES

FIG. 1 illustrates a four-part 2D side-by-side view of an absorbent article.

FIG. 2 illustrates a five-part 2D side-by-side view of an absorbent article.

FIG. 3 illustrates a five-part exploded view of an absorbent article.

FIG. 4 illustrates a six-part exploded view of an absorbent article.

FIG. 5 illustrates a front view and a top view of three garments, each with an absorbent article.

### DETAILED DESCRIPTION

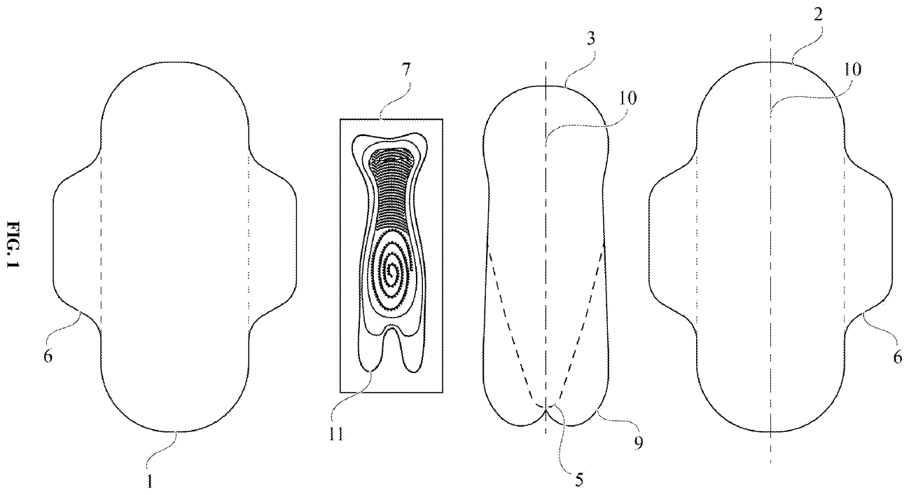

The present invention, and referring to FIG. 1, corresponds to an adjustable absorbent article comprising a topsheet (1), a bottom sheet (2), an absorbent core (3) located between the top sheet (1) and the bottom sheet (2), the absorbent core (3) having an oblong shape with two longitudinal ends. One of the longitudinal ends has a triangular shape and, in addition, the absorbent core (3) has a section corresponding to an absorbent section (9) that extends from the periphery of the triangular shaped longitudinal end towards the exterior of the absorbent article. Said absorbent section (9) folds to fit different types of garments.

The topsheet (1) of the adjustable absorbent article is a permeable sheet that allows absorbing and retaining fluids that come into contact with said topsheet (1). The top sheet (1) also allows the skin of a user not to be irritated when in contact with it. Said topsheet (1) is made of a permeable material.

Additionally, the topsheet (1) optionally is composed of two or more layers of permeable material, which allows controlling permeability of the fluids that come into contact with said sheet due to the number of layers that can make up the topsheet (1).

Moreover, the bottom sheet (2) that is connected to the absorbent core (3), or in other words, the bottom sheet (2) that is located below the absorbent core (3), is fluid impervious and is in contact with user garments. Said bottom sheet (2) prevents fluids absorbed by the absorbent core (3) from contacting the surface of a garment with which the bottom sheet (2) is in contact with.

Preferably, the top sheet (1) and the bottom sheet (2) have the same geometric shape, allowing them to be joined by their contour by means of a binding material, heat or an adhesive material. In addition, the surface of the bottom sheet (2) that is in contact with a user garment optionally has an adhesive material on its surface that allows the adjustable absorbent article to be attached to the garment.

The waterproof material of the bottom sheet (2) is selected from the group consisting of polyethylene, polypropylene, polyvinylchloride (PVC); chlorinated polyvinyl chloride (CPVC); polyethylene terephthalate (PET), polyamides (PA) (for example, PA12, PA6, PA66); polychlorotrifluoroethylene (PCTFE); vinylidene polyfluoride (PVDF), fiber reinforcements (for example, glass, aramid, polyester), ethylene polytetrafluoride (PTFE); ethylene-chlorotrifluoroethylene (ECTFE); plastics (polyester, vinyl ester, epoxy, vinyl resins), thermoplastic films, non-woven textiles, materials known to any skilled person in the art and combinations thereof.

For the better understanding of the present invention, a person, user, or end user is understood to be that person who uses a garment on which an absorbent article can be placed.

Moreover, and referring to FIG. 1, the absorbent core (3) located between the top sheet (1) and the bottom sheet (2), has an oblong shape with two longitudinal ends, where one of the longitudinal ends has a triangular shape. Said triangular-shaped longitudinal end allows the absorbent section (9) to fold over and around one of the edges of said triangular shape. One of the effects of the foregoing is that a portion of the absorbent section (9) folds over a garment, allowing the adjustable absorbent article to be attached to different types of garments such as thong-style garments, pantyhose-style garments, bikinis, regular panties, high cut panties, boyshorts, boxers and body shapers. Furthermore, it is not necessary for the entire absorbent section (9) to be folded over the garments in order for the section to be adequately fixed on the garment.

Additionally, the triangular shape of the longitudinal ends, instead of having a vertex, optionally has a shape selected from the group consisting of: squares, triangles, circles, rectangles, pentagons, trapezoids, ellipses, rhombs, trapezoids, hexagon, heptagon, octagon, decagon, and equivalent shapes known to a skilled person in the art and combinations thereof.

On the other hand, the absorbent core (3) is optionally formed by more than one layer or more than one material, which allows controlling the absorption of fluids that come into contact with the adjustable absorbent article due to the number of layers the absorbent core (3) can have or the combination of materials.

The absorbent core material (3) is selected from the group consisting of cellulosic fibers (preferably wood pulp, although cotton, linen (flax) and peat moss are acceptable), synthetic fibers, superabsorbent polymers (SAP) or superabsorbent fibers, as well as organic binders, or equivalent materials known to a skilled artisan and combinations thereof. The ratio of the aforementioned materials may change to achieve sufficient absorption, compressibility and processability.

Moreover and referring to FIG. 1, the absorbent section (9) extending from the periphery of the triangular-shaped longitudinal end of the absorbent core (3), is a layer of an absorbent material. Said absorbent section (9) optionally has an adhesive material on the face that is in contact with a user's garment.

Preferably, the absorbent section (9) has a thickness less than the thickness of the absorbent core (3). One of the purposes of the above is to allow for the absorbent section (9) to be folded without generating discomfort for the user when the thickness of said absorbent section (9) is added to the thickness of the absorbent core (3). Another technical effect generated by the fact that the absorbent section (9) is less thick than the absorbent core (3) is allowing the absorbent section (9) to be completely folded into a garment, and ergonomically shaped for a user.

Referring to FIG. 1, there is a longitudinal axis (10) between the two longitudinal ends of the absorbent core (3). Preferably, the longitudinal axis (10) is located in the middle of the adjustable absorbent article. On the other hand, the adjustable absorbent article comprises a transverse axis, perpendicular to the longitudinal axis (10) and located in the middle of the adjustable absorbent article. Additionally, and in an embodiment of the invention, the top sheet (1), the bottom sheet (2) and the absorbent core (3) are symmetrical with respect to the longitudinal axis (10).

In one embodiment of the invention, and referring to FIG. 1, the absorbent article has two absorbent sections (9). Each of the absorbent sections (9) extend from the periphery of the triangular-shaped longitudinal end of the absorbent core (3). A first absorbent section is located on one side of the longitudinal axis (10), and a second absorbent section is located on the opposite side of the longitudinal axis (10). One of the technical effects generated is allowing said absorbent sections (9) to be fastened at two points of a user garment, which allows for a better grip of the adjustable absorbent article.

Each of the absorbent sections (9) may be made of different materials, or different materials than the absorbent core (3), but preferably they are of the same material to warrant the same liquid absorption. In said embodiment of the invention, the second absorbent section is symmetrical with respect to the longitudinal axis (10), and to the first absorbent section. Additionally, and in said embodiment of the invention, the adjustable absorbent article is also symmetrical with respect to the longitudinal axis (10), which allows it to fit symmetrically on different types of garments and does not create user discomfort.

Referring to FIG. 1, the triangle-shaped vertex of the longitudinal end has a rounded vertex (5), where said rounded vertex (5) allows covering more area of the user garment, in turn allowing the adjustable absorbent article to adjust to different types of garments. Moreover, the absorbent section (9) can be tangent to the rounding of the rounded vertex (5) and be positioned adjacent to it, allowing the adjustable absorbent article to be attached to garments where the longitudinal ends of the adjustable absorbent article are located in areas where the width of a garment is less than the width of the absorbent core (3).

On the other hand, the absorbent section (9) can also be distal to the curvature of the rounded vertex (5), allowing the adjustable absorbent article to be adjusted when a garment has a width equal to or greater than the width of the absorbent article where it is positioned.

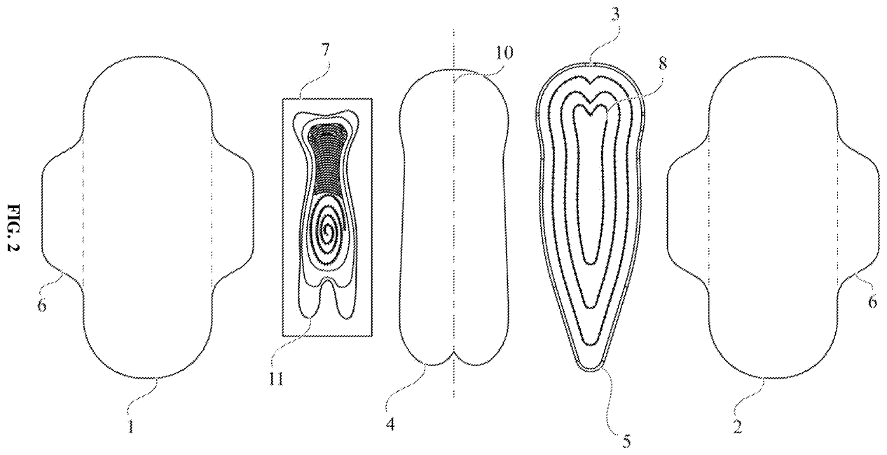

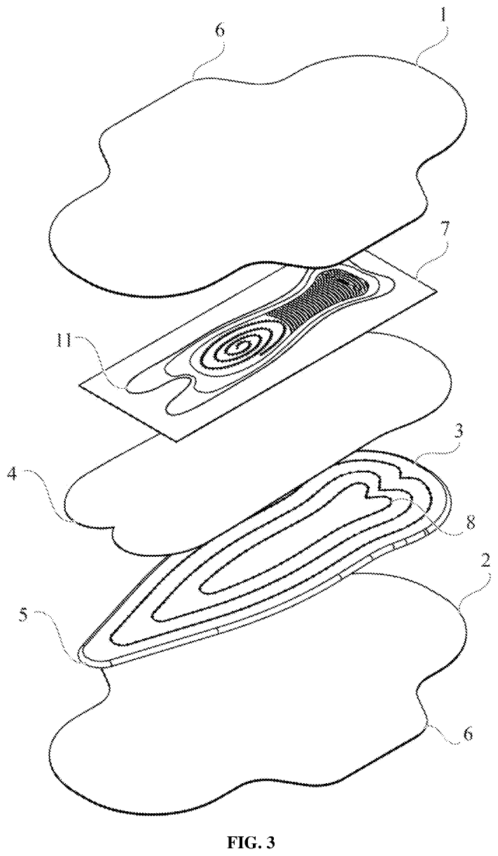

On the other hand, in another embodiment of the invention and referring to FIG. 2 and FIG. 3, the adjustable absorbent article comprises: a top sheet (1), a bottom sheet (2) and an absorbent core (3) located between the top sheet (1) and the bottom sheet (2), wherein said absorbent core (3) has an oblong shape and has two longitudinal ends. Said absorbent core (3) has a triangular shape at one of the longitudinal ends, and an absorbent panel (4) overlapping and protruding on the periphery of the triangular-shaped longitudinal end. Said absorbent panel (4) folds to fit different types of garments.

In said embodiment of the invention, the absorbent core (3) located between the top sheet (1) and the bottom sheet (2), has an oblong shape with two longitudinal ends, where one of the longitudinal ends has a triangular shape. Said triangular-shaped longitudinal end allows the absorbent panel (4) to fold over one of the edges of said triangular shape. One of the technical effects of folding the absorbent panel (4) over one of said edges is that it can be folded on a garment. This allows the adjustable absorbent article to be attached to different types of garments such as thong garments, pantyhose garments, bikini bottoms, normal panties, high cut panties, boyshorts and body shapers. Furthermore, it is not necessary for the entire protruding absorbent panel (4) to be folded over the garments in order for the panel to be adequately fixed on the garment.

Moreover, and referring to FIG. 2 and FIG. 3, the absorbent panel (4) overlapped on the periphery of the triangular-shaped longitudinal end of the absorbent core (3), is a layer of an absorbent material. The absorbent panel (4) optionally has an adhesive material on the face that is in contact with the user garment.

Optionally, the absorbent panel (4) has a thickness less than the thickness of the absorbent core (3). One of the purposes of the above is to allow the absorbent panel (4) to be folded without generating discomfort for the user when the thickness of said absorbent panel (4) is added to the thickness of the absorbent core (3). Another technical effect generated by the fact that the absorbent panel (4) is less thick than the absorbent core (3), is allowing the absorbent panel (4) to be completely folded into a garment, and ergonomically shaped for a user.

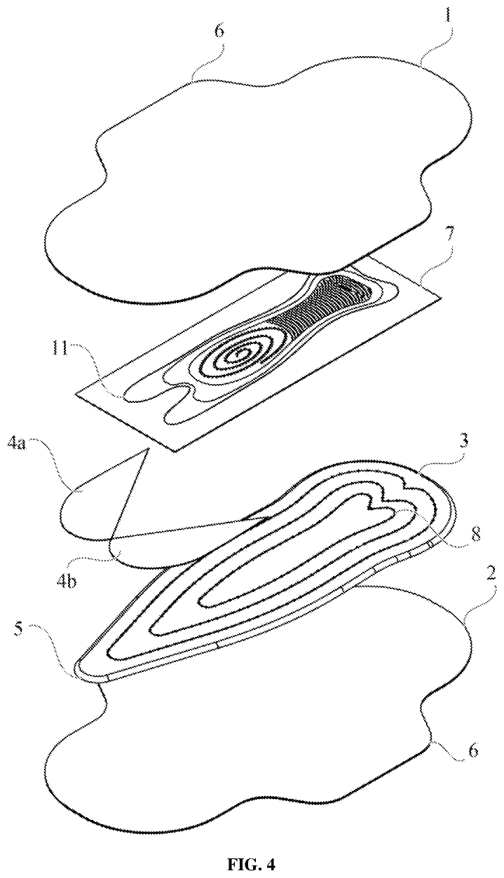

In one embodiment of the invention, and referring to FIG. 4, the absorbent article has two absorbent panels (4), a first absorbent panel (4a) located on one side of the longitudinal axis (10) on the triangular-shaped longitudinal end, and a second absorbent panel (4b), located on the opposite side of the longitudinal axis (10) on the triangular-shaped longitudinal end. One of the technical effects generated is allowing said absorbent panels (4a and 4b) to be fastened at two points of a user's garment when they are folded, which allows for a better grip of the adjustable absorbent article. This happens given that the bottom sheet (2) also folds when the absorbent panels (4) are folded, allowing for the adhesive of the bottom sheet (2) be fixed to the user garment.

Each of the absorbent panels (4a and 4b) can be made of different materials, or they can be of the same material to warrant the same liquid absorption. Preferably the second absorbent panel (4b) is symmetrical to the first absorbent panel (4a) with respect to the longitudinal axis (10), and the adjustable absorbent article is also symmetrical with respect to the longitudinal axis (10), allowing it to be symmetrically adjusted to different types of garments.

Referring to FIG. 2 and FIG. 3, the triangle-shaped vertex of the longitudinal end has a rounded vertex (5), where said rounded vertex (5) allows for more user comfort. Moreover, the absorbent panel (4) can be located tangent to the rounding of the rounded vertex (5) and be positioned adjacent to it, allowing the adjustable absorbent article to be attached to garments where the longitudinal ends of the adjustable absorbent article are located within the narrowest areas of undergarment reinforcement.

On the other hand, the absorbent panel (4) can also be distal to the curvature of the rounded vertex (5), which allows the adjustable absorbent article to be adjusted when a garment has a thickness less than the vertex of the triangular-shaped longitudinal end of the absorbent core (3).

On the other hand, and referring to FIG. 1, FIG. 2, FIG. 3 and FIG. 4 the adjustable absorbent article comprises an acquisition layer (7) located between the top sheet (1) and the bottom sheet (2). Preferably, the acquisition layer (7) extends over the absorbent core (3) and the absorbent panel (4), or it can also be extended over the absorbent core (3) and the absorbent section (9). Said acquisition layer (7) makes it possible to distribute the fluids that come into contact with the topsheet (1) along the surface of the adjustable absorbent article. Preferably, and referring to FIG. 1, the acquisition layer (7) is located between the topsheet (1) and the absorbent core (3). This allows the fluid that comes into contact with the topsheet (1) to be distributed along the absorbent core (3), which allows for uniform absorption of said fluid.

Additionally, and referring to FIG. 2, FIG. 3, and FIG. 4, the adjustable absorbent article is made up of a top sheet (1), a bottom sheet (2), an absorbent core (3) located between the top sheet (1) and the bottom sheet (2), an absorbent panel (4) located between the topsheet (1) and the absorbent core (3), and an acquisition layer (7) located between the topsheet (1) and the absorbent panel (4). This allows the acquisition layer (7) to distribute the fluid that comes into contact with the adjustable absorbent article, along the absorbent panel (4) and in turn, the absorbent core (3).

Further, the adjustable absorbent article has at least one wing (6), wherein said wing (6) is located on the periphery of the adjustable absorbent article. Preferably, the wing (6) is on the top sheet (1) or on the bottom sheet (2), and is made of the same material as the top sheet (1) or the bottom sheet (2). Optionally, the surface of the wing (6) that is in contact with a user garment, has an adhesive material that allows the adjustable absorbent article to be attached to said garment.

In one embodiment of the invention, and referring to FIG. 2 and FIG. 3, the adjustable absorbent article comprises at least two wings (6), each located on the periphery of the topsheet (1) and the periphery of the bottom sheet (2) and extending towards the outside of the adjustable absorbent article. Preferably each of the wings (6) is on one side of the longitudinal axis (10), while the other wing (6) is on the opposite side of the longitudinal axis (10) and both can be arranged symmetrically.

Moreover, the absorbent core (3) preferably has absorbent granules (12). Said absorbent granules (12) are a particulate material embedded in the absorbent core (3), which allow the adjustable absorbent article to absorb more fluid. Said absorbent granules (12) can also be located in the top sheet (1), the bottom sheet (2), the absorbent panel (4) or the wings (6). Said absorbent granules (12) allow increasing the absorption of fluid throughout the adjustable absorbent article, that allows spill reduction.

The absorbent granules (12) are selected from the group consisting of sodium polyacrylate, potassium polyacrylate, silica gel, hydrogel, inorganic or organic crosslinked hydrophilic polymers, such as polyvinyl alcohols, polyethylene oxides, crosslinked starches, guar gum, xanthan gum, equivalent materials known to any skilled artisan or combinations thereof.

On the other hand, and preferably, the absorbent core (3), the top sheet (1), the bottom sheet (2), the absorbent panel (4) or the acquisition layer (7) have at least one marking (8). Said marking (8) are embossed and debossed areas that allow distributing fluids that come into contact with the adjustable absorbent article along its entire surface. In addition, said marking (8) also allows said fluids that come into contact with the adjustable absorbent article, to not extend beyond the periphery of the absorbent core (3). Referring to FIG. 2, FIG. 3 and FIG. 4, absorbent core (3) has a marking (8) on its surface.

The shape of the marking (8) is selected from the group consisting of curves, curved lines having several turns around a point, spirals, hyperbolic spirals, Archimedean spirals, logarithmic spirals, Fermat spirals, squares, triangles, circles, rectangles, pentagons, trapezoids, ellipses, rhombuses, hexagons, heptagons, octagons, decagons, and equivalent shapes known to a skilled artisan or combinations thereof.

On the other hand, any of the faces of the absorbent core (3), the top sheet (1), the bottom sheet (2), the absorbent panel (4) or the acquisition layer (7) optionally have an imprint (11) on its surface. Said imprint (11) preferably has a shape that gives a feeling of depth, wherein one of the technical effects of including said imprint (11) is to generate in the end user a perception of depth which allows the person using the adjustable absorbent article to feel more confident when wearing it.

The imprint (11) is selected from the group consisting of curves, curved lines having several turns around a point, spirals, hyperbolic spirals, Archimedean spirals, logarithmic spirals, Fermat spirals, squares, triangles, circles, rectangles, pentagons, trapezoids, ellipses, rhombuses, hexagons, heptagons, octagons, decagons, and equivalent forms known to a skilled artisans or combinations thereof.

### Example 1

Referring to FIG. 1, the adjustable absorbent article is made up of a top sheet (1), a bottom sheet (2), an absorbent core (3) located between the top sheet (1) and the bottom sheet (2) and an acquisition layer (7) located between the topsheet (1) and the absorbent core (3). The top sheet (1) and the bottom sheet (2) have the same shape, corresponding to one oval formed by two semi circles joined by a rectangle. Both the top sheet (1) and the bottom sheet (2) have two wings (6), each one located on the straight edge of the rectangle that makes up each of the sheets (1, 2).

Moreover, the absorbent core (3) has an oblong shape with two longitudinal ends, wherein one of the longitudinal ends has a triangular shape and a rounded vertex. Additionally, said absorbent core (3) has two overlapping absorbent sections (9) on the periphery of the triangular-shaped longitudinal end of the absorbent core (3). Said adjustable absorbent article also has an imprint (11) located on the acquisition layer (7).

### Example 2

Referring to FIG. 2, FIG. 3, the adjustable absorbent article is made up of a top sheet (1), a bottom sheet (2), an absorbent core (3) located between the top sheet (1) and the bottom sheet (2), an absorbent panel (4) located between the topsheet (1) and the absorbent core (3), and an acquisition layer (7) located between the topsheet (1) and the absorbent panel (4). The topsheet (1) and the bottom sheet (2) have the same shape, corresponding to an oval formed by two semi circles joined by a rectangle. Both the topsheet (1) and the bottom sheet (2) have two wings (6), each one arranged on the straight edge of the rectangle that make up each of the sheets (1, 2).

Moreover, the absorbent core (3) has an oblong oval shape with two longitudinal ends, wherein one of the longitudinal ends has a triangular shape and a rounded vertex. Additionally, said absorbent core (3) has a marking (8) on its surface. Said adjustable absorbent article also has an imprint (11) located on the acquisition layer (7).

### Example 3

Referring to FIG. 4, the adjustable absorbent article is made up of a top sheet (1), a bottom sheet (2), an absorbent core (3) located between the top sheet (1) and the bottom sheet (2), two absorbent panels, one first absorbent panel (4a) and a second absorbent panel (4b), both located between the topsheet (1) and the absorbent core (3), and an acquisition layer (7) located between the topsheet (1) and the absorbent panel (4). The top sheet (1) and the bottom sheet (2) have the same shape, corresponding to an oval formed by two semi circles joined by a rectangle. Both the top sheet (1) and the bottom sheet (2) have two wings (6), each one located on the straight edge of the rectangle that make up each of the sheets (1, 2).

Moreover, the absorbent core (3) has an oblong shape with two longitudinal ends, wherein one of the longitudinal ends has a triangular shape and a rounded vertex, wherein the absorbent panels (4a, 4b) are overlapped on the edges of the triangular-shaped end. Additionally, said absorbent core (3) has a marking (8) on its surface. Said adjustable absorbent article also has an imprint (11) located on the acquisition layer (7).

### Example 4

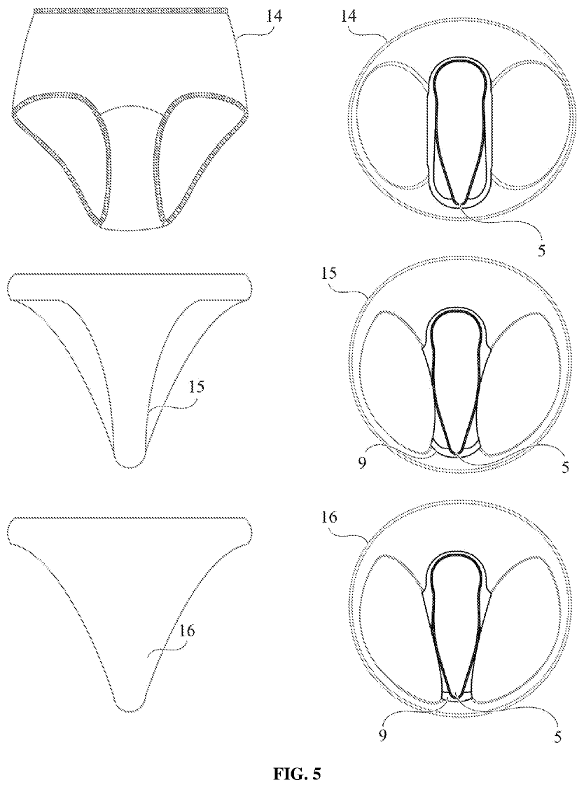

Referring to FIG. 5, three types of underwear are illustrated: namely, a normal pantie style garment (14), a pantie garment (15) and a thong style garment (16), wherein each of the garments has the adjustable absorbent article of example 3. The adjustable absorbent article positioned in the normal pantie style garment (14), only has folds on the wings (6). This is because the area width of the normal pantie style garment (14), where the adjustable absorbent article is found, is equal to the width of the adjustable absorbent article without wings (6).

Moreover, the adjustable absorbent article positioned on the pantie-style garment (15), has folds both on the wings (6) and on the absorbent panel (4), because the area width of the pantie-style garment (15), where the adjustable absorbent article is found, is less than the width of the wingless adjustable absorbent article (6). This allows the adjustable absorbent article to be fixed to the garment in 4 support points.

Finally, the adjustable absorbent article positioned on the thong-like garment (16) has folds on the wings (6), and the absorbent panel (4) folds completely. This is due to the fact that the width of the area of the thong-style garment (16), where the adjustable absorbent article is found, is equal in size to the triangular shape of the absorbent article.

It should be understood that the present invention is not limited to the illustrated embodiments, given any skilled artisan will find variations and possible modifications that do not depart from the spirit of the invention, which are only defined by the following claims.

What is claimed is:

1. An adjustable absorbent article comprising:
a top sheet;
a bottom sheet;
an absorbent core located between the top sheet and the bottom sheet, the absorbent core having an oblong shape with two longitudinal ends, wherein one of the longitudinal ends has a triangular shape; and
an absorbent panel overlapped on the periphery of the triangular-shaped longitudinal end of the absorbent core;
wherein the absorbent panel folds to fit different types of garments.

2. The article according to claim 1 characterized in that the triangular-shaped longitudinal end has a rounded vertex.

3. The article according to claim 1 characterized in that the absorbent panel has a thickness less than the thickness of the absorbent core.

4. The article according to claim 1 characterized in that between the two longitudinal ends of the absorbent core there is a longitudinal axis, wherein the adjustable absorbent article comprises a first absorbent panel located on one side of the longitudinal axis on the triangular-shaped longitudinal end, and a second absorbent panel located on the other side of the longitudinal axis on the triangular-shaped longitudinal end and symmetrical to the first absorbent panel.

5. The article according to claim 1 characterized in that there is an acquisition layer between the top sheet and the bottom sheet.

6. The article according to claim 1 characterized in that the absorbent article has wings.

7. The article according to claim 1 characterized in that the absorbent core has absorbent granules.

8. The article according to claim 1 characterized in that any of the absorbent core, top sheet, bottom sheet and absorbent panel has at least one marking.

9. The article according to claim 1 characterized in that any of the absorbent core, top sheet, bottom sheet and absorbent panel has at least one imprint.
